(19) United States
(12) Patent Application Publication
(10) Pub. No.: US 2022/0031512 A1
(43) Pub. Date: Feb. 3, 2022

(54) SYSTEMS AND METHODS FOR EYE CATARACT REMOVAL

(71) Applicant: Alcon Inc., Fribourg (CH)

(72) Inventors: David Thoe, Laguna Hills, CA (US); Brant Gillen, Southlake, TX (US); George Hunter Pettit, Fort Worth, TX (US); Marcos H. Zamorano, Fort Worth, TX (US); Keith Watanabe, Irvine, CA (US); Ramesh Sarangapani, Coppell, TX (US); Sinchan Bhattacharya, Denton, TX (US); Joseph Weatherbee, San Clemente, CA (US)

(21) Appl. No.: 17/380,992

(22) Filed: Jul. 20, 2021

Related U.S. Application Data
(60) Provisional application No. 63/059,355, filed on Jul. 31, 2020.

(51) Int. Cl. A61F 9/008; A61F 9/007; A61B 3/117

(52) U.S. Cl. CPC A61F 9/00834; A61F 9/00745; A61F 2009/00878; A61F 2009/00887; A61B 3/1173

(57) ABSTRACT

Systems and methods for assisting in the removal of a cataract from an eye can include obtaining pre-operative data for the eye, the pre-operative data including imaging data associated with the lens of the eye, determining a lens density map based on the imaging data associated with the lens, and generating laser fragmentation patterns for a laser fragmentation procedure based on the lens density map.

### BACKGROUND

### Field of the Disclosure

The present disclosure relates to systems and methods for removal of a cataract from an eye.

### Description of Related Art

Cataract surgery involves removing the natural lens of an eye and, in most cases, replacing the natural lens with an artificial intraocular lens (IOL). Typically, removal of the natural lens involves phacoemulsification, which is a surgical practice of using an ultrasonic handpiece to emulsify the patient's natural lens and aspirate the emulsified lens material from the eye. In some cases, a patient and a surgeon will elect laser-assisted surgery, which involves using a laser (e.g. femtosecond laser) to make incisions in the lens capsule, fragment and soften the cataract, create limbal relaxing incisions (LRI), perform astigmatic keratotomy (AK), etc.

To achieve an optimal post-operative visual outcome, a good pre-operative surgical plan is crucial. Some of the important pre-operative planning decisions involve the selection of appropriate patterns and/or settings for the laser, phacoemulsification, and/or other equipment to be used to remove the cataract from the eye prior to the implantation of the IOL. Given the complexity of the procedure and the variability in possible patterns and/or settings for the laser, the phacoemulsification, and/or the other equipment, planning and performance of the cataract removal procedure may be challenging. In addition, the variability between different patients (e.g., health history factors, etc.), different eyes, different cataracts (e.g., shape, density, etc.), and/or the like, further compounds the complexity of the planning and performance of the cataract removal.

### SUMMARY

Some embodiments of the present technology involve systems, computer-readable media, and methods for obtaining pre-operative data for the eye, the pre-operative data including imaging data associated with the lens of the eye, determining a lens density map based on the imaging data associated with the lens, and generating laser fragmentation patterns for a laser fragmentation procedure based on the lens density map.

Also described herein are embodiments of a non-transitory computer readable medium comprising instructions to be executed in a system, wherein the instructions when executed in the system perform the method described above.

Also described herein are embodiments of a system, wherein software for the system is programmed to execute the method described above.

Also described herein are embodiments of a system comprising means for executing the method described above.

### BRIEF DESCRIPTION OF THE DRAWINGS

For a more complete understanding of the present technology, its features, and its advantages, reference is made to the following description, taken in conjunction with the accompanying drawings.

FIG. 1 is a diagram of an example system for eye surgery according to some embodiments.

FIGS. 2A-2B show a diagram of a method of removing a cataract according to some embodiments.

FIG. 3 is a diagram of an eye and characteristics of the eye according to some embodiments.

FIGS. 4A and 4B are diagrams of processing systems according to some embodiments.

FIG. 5 is a diagram of a multi-layer neural network according to some embodiments.

In the figures, elements having the same designations have the same or similar functions.

### DETAILED DESCRIPTION

This description and the accompanying drawings that illustrate inventive aspects, embodiments, implementations, or modules should not be taken as limiting—the claims define the protected invention. Various mechanical, compositional, structural, electrical, and operational changes may be made without departing from the spirit and scope of this description and the claims. In some instances, well-known circuits, structures, or techniques have not been shown or described in detail in order not to obscure the invention. Like numbers in two or more figures represent the same or similar elements.

In this description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

Before a more detailed discussion of the systems, methods, prediction models, optimized surgical plans, etc., a brief discussion of the technical problems the present technology solves is provided. As explained above, cataract surgery involves removing the natural lens of an eye and, in most cases, replacing the natural lens with an artificial intraocular lens (IOL). Typically, removal of the natural lens involves phacoemulsification, which is a surgical practice of using an ultrasonic handpiece to emulsify the patient's natural lens and aspirate the emulsified lens material from the eye. In some cases, a patient and a surgeon will elect laser-assisted surgery, which involves using a laser (e.g., femtosecond laser) to make incisions in the lens capsule, fragment and soften the cataract prior to phacoemulsification, create limbal relaxing incisions (LRI), perform astigmatic keratotomy (AK), etc.

When performing laser-assisted surgery, the patient is fitted with a patient adaptor, which is placed on the eye and which uses suction on the eye to maintain alignment with the laser. In some cases, one of the goals in planning a laser-assisted surgery is to reduce the time the patient's eye is under suction. In some cases, another goal is to reduce the amount of laser energy that is delivered to portions of the eye (e.g., to reduce or eliminate gas bubbles created as an unwanted side effect of the laser energy, which can lead to less than optimal surgical outcomes). Also, oftentimes a surgeon has a preferred pattern for phacoemulsification and aspiration of lens material. For example, a surgeon may have been trained to complete phacoemulsification and aspiration of lens material in a certain repeatable pie-slice pattern. The surgeon can be accustomed to emulsifying and removing lens material from a first pie slice and rotating around the lens to subsequent slices to ensure that each area is adequately emulsified and aspirated.

In addition to minimizing time under suction and the total amount of laser energy, in certain embodiments, it may be advantageous to minimize the number of laser spots, minimize the total length of laser pattern lines, minimize the time required for phacoemulsification, minimize the total ultrasonic energy required for phacoemulsification, minimize the time required to aspirate the lens, minimize the amount of fluid required for aspiration, and/or a wide variety of other optimization criteria and surgeon preferences described in more detail below.

However, existing ophthalmic systems (e.g., ophthalmic surgical and/or diagnostic systems) are not configured to automatically optimize for these parameters in preparation for or during cataract surgery, thereby, leading to inefficient use of resources, such as laser energy, ultrasonic energy, compute and memory resources of surgical systems and consoles, amount of fluid required for aspiration, etc.

Accordingly, certain embodiments described herein provide technical solutions to the technical problems associated with existing ophthalmic systems by obtaining pre-operative diagnostic images and/or other data for a patient and, e.g., automatically, providing a recommended fragmentation pattern, recommended laser settings, recommended phacoemulsification settings, etc., based on the pre-operative data. The recommendations may be configured to optimize for the parameters described above, thereby not only resulting in resource efficiency but also more satisfactory patient outcomes.

For example, pre-operative images of a patient's eye can be used to create a lens density map for the patient's eye(s). Then, trained prediction models (e.g., trained based on historical patient data, historical time under suction metrics, historical laser energy metrics, quantified surgical outcome metrics, etc.) may use, as input, the lens density map to recommend a fragmentation pattern, laser settings, phacoemulsification settings, etc., in order to optimize the surgical outcome for a current patient. In particular, in some embodiments of the present technology, a recommended fragmentation pattern can conform with a surgeon's routine, repeatable pattern (e.g., pie-slice pattern). In addition, the surgical plan provided by the present technology can include recommendations for which slices to treat with laser energy, how much laser energy to dedicate to each slice, how much ultrasonic power to deliver to each area of each slice (e.g., based on a predicted ultrasonic power required after the recommended amount of laser energy is delivered to the particular slice), etc. In some other cases, an optimized surgical plan can recommend a custom fragmentation pattern and device settings based on the lens density map and on various surgical optimization criteria (e.g., reduction of time under suction, reduction of total laser energy, reduction of total ultrasonic power, etc.) that are selected by a surgeon and/or recommended by the prediction model.

Planning for cataract surgery typically also involves selecting an IOL power with the goal of achieving a desired refractive outcome (interchangeably referred to as a refractive target) post-surgery. Certain embodiments described herein provide systems and techniques for assisting a surgeon with selecting an IOL with an optimal IOL power. For example, certain embodiments described herein involve receiving pre-operative and/or intra-operative measurements from a patient's eye(s) and estimating a post-operative manifest refraction in spherical equivalent (MRSE), e.g., for each of a given set of IOL powers. Using the post-operative MRSEs, the surgeon may then select the IOL power that results in an estimated post-operative MRSE that is closest to the refractive target. Examples of these techniques are described in further detail in U.S. Pat. Ser. No. 62/697,367 disclosing “OPHTHALMIC IMAGING SYSTEM FOR INTRAOCULAR LENS POSITION AND POWER SELECTION” and U.S. patent Ser. No. 16/171,515 disclosing “SYSTEMS AND METHODS FOR INTRAOCULAR LENS SELECTION USING EMMETROPIA ZONE PREDICTION”, both of which are hereby incorporated by reference in their entirety.

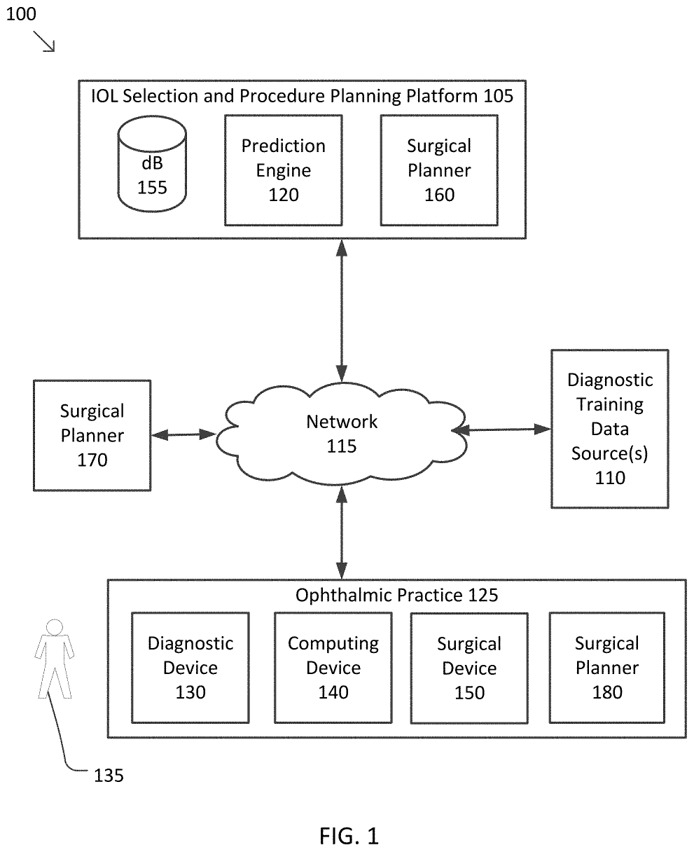

With the above examples and considerations in mind, FIGS. 1-6 provide more detail on the systems and methods for assisting eye cataract removal according to some embodiments of the present technology.

FIG. 1 illustrates a system 100 for eye surgery according to some embodiments. The system 100 includes an IOL selection and procedure planning platform 105 (hereinafter “ISP platform 105”) coupled with one or more diagnostic training data sources 110 via a network 115. In some examples, the network 115 may include one or more switching devices, routers, local area networks (e.g., an Ethernet), wide area networks (e.g., the Internet), and/or the like. Each of the diagnostic training data sources 110 may be a database, a data repository, and/or the like made available by an ophthalmic surgery practice, an eye clinic, a medical university, an electronic medical records (EMR) repository, and/or the like. Each of the diagnostic training data sources 110 may provide ISP platform 105 with training data in the form of one or more of multi-dimensional images and/or measurements of patients' pre- and post-operative eyes, surgical planning data, surgical console parameter logs, surgical complication logs, patient medical history, patient demographic data, information on an implanted IOL, patient preferences (e.g., ability to drive at night, ability to read without glasses, etc.) and/or the like. The ISP platform 105 may store the training data in one or more databases 155 which may be configured to anonymize, encrypt, and/or otherwise safeguard the training data.

The ISP platform 105 includes a prediction engine 120 which may process the received training data, perform raw data analysis on the training data, and train and iteratively optimize one or more machine learning models (interchangeably referred to as prediction models). The trained machine learning models may be used to assist in the planning and performance of a surgical procedure (e.g., cataract removal, IOL implantation, and/or the like). For example, based on the patient's pre-operative measurements, the prediction engine 120 may generate a custom and optimized surgical plan that includes recommended patterns and/or device settings for the surgical procedure, and estimated post-operative MRSEs, e.g., for each of a given set of IOL powers. Note that herein, the recommended patterns and device settings may include a recommended fragmentation pattern, recommended laser settings, recommended phacoemulsification settings, recommendations for which slices to treat with laser energy, how much laser energy to dedicate to each slice, how much ultrasonic power to deliver to each area of each slice (e.g., based on a predicted ultrasonic power required after the recommended amount of laser energy is delivered to the particular slice).

In some examples, the machine learning models (e.g., one or more neural networks) are trained at least in part based on pre-operative measurements and corresponding intra-operative measurements and/or post-operative outcomes obtained from the one or more diagnostic training data sources 110. As an example, eye care professionals can take efforts to quantify surgical outcomes. For example, a wide collection of surgical parameters and pre-, intra-, and post-operative diagnostic can be gathered for a group of patients and the patients can be given a post-operative satisfaction survey. The results of the survey can be used to train a computational model to train machine learnings models for optimizing settings, techniques, materials for future procedures. Examples of this technique are described in greater detail in U.S. Provisional Patent Application No. 63/032,195, entitled “SELECTION OF INTRAOCULAR LENS BASED ON PREDICTED SUBJECTIVE OUTCOME SCORE”, which is incorporated by reference in its entirety.

The ISP platform 105 is further coupled, via network 115, to one or more devices of an ophthalmic practice 125. The one or more devices include one or more diagnostic devices 130. The one or more diagnostic devices 130 are used to obtain one or more multi-dimensional images and/or other measurements of an eye of a patient 135. The one or more diagnostic devices 130 may be any of a number of devices for obtaining multi-dimensional images and/or measurements of ophthalmic anatomy such as an optical coherence tomography (OCT) device, a rotating camera (e.g., a Scheimpflug camera), a magnetic resonance imaging (MRI) device, a keratometer, an ophthalmometer, an optical biometer, a three-dimensional stereoscopic digital microscope (such as NGENUITY® 3D Visualization System (Alcon Inc., Switzerland), any type of intra-operative optical measurement device, such as an intra-operative aberrometer, and/or any other type of optical measurement/imaging device. Examples of OCT devices are described in further detail in U.S. Pat. No. 9,618,322 disclosing “Process for Optical Coherence Tomography and Apparatus for Optical Coherence Tomography” and U.S. Pat. App. Pub. No. 2018/0104100 disclosing “Optical Coherence Tomography Cross View Image”, both of which are hereby incorporated by reference in their entirety. An example of an intra-operative aberrometer is Ora™ with Verifeye™ (Alcon Inc., Switzerland), which is partially described in more detail in commonly owned U.S. Pat. No. 7,883,505 disclosing “Integrated Surgical Microscope and Wavefront Sensor” and U.S. Pat. No. 8,784,443 disclosing “Real-Time Surgical Reference Indicium Apparatus and Methods for Astigmatism Correction”, both of which are hereby incorporated by reference in their entirety.

The ophthalmic practice 125 may also include one or more computing devices 140 for obtaining, from the one or more diagnostic devices 130, the multi-dimensional images and/or measurements of patient 135 and sending them to the ISP platform 105. The one or more computing devices 140 may be one or more of a stand-alone computer, a tablet and/or other smart device, a surgical console, a computing device integrated into the one or more diagnostic devices 130, and/or the like.

The ISP platform 105 may receive data relevant to patient 135 (e.g., measurements, images, etc.), which is then utilized by the prediction engine 120 to generate a custom and optimized surgical plan for the patient, thereby assisting in the planning and performance of cataract surgery for the patient. For example, as described above, the prediction engine 120 may generate recommended fragmentation patterns and/or device settings for cataract removal. The prediction engine 120 may further help the user select an IOL by providing the user with post-operative MRSE estimates for different IOL powers. Therefore, by providing the different types of outputs described above, the prediction engine 120 helps with improving post-operative patient outcomes. In addition, configuring an ophthalmic system, such as system 100, to automatically provide recommended fragmentation patterns and/or device settings as well as, e.g., targets, and/or feedback to allow a surgeon to perform the surgery based on the recommended fragmentation patterns and/or device settings improves the technical field of ophthalmic surgery as well as the ophthalmic system itself, which includes ophthalmic surgical systems and consoles (e.g., surgical device 150).

The ophthalmic practice 125 may also include one or more surgical devices 150 to perform one or more procedures on an eye, such as cataract removal, IOL implantation, and/or the like. The one or more surgical devices 150 may include a laser system for pre-fragmentation of a cataract, such as the laser systems described in more detail in commonly owned U.S. Pat. No. 9,427,356 disclosing “Photodisruptive Laser Fragmentation of Tissue” and U.S. Pat. No. 9,622,913 disclosing “Imaging-Controlled Laser Surgical System”, both of which are hereby incorporated by reference in their entirety. The one or more surgical devices 150 may further include a phacoemulsification device for using ultrasonics and fluidics to further fragment and remove the cataract from the eye, such as the phacoemulsification system described in more detail in commonly owned U.S. Pat. No. 8,939,927 disclosing “Systems and Methods for Small Bore Aspiration”, which is hereby incorporated by reference in its entirety. The one or more surgical devices 150 may also refer to surgical consoles that incorporate a laser system, a phacoemulsification device, and/or other components for performing additional ophthalmic procedures.

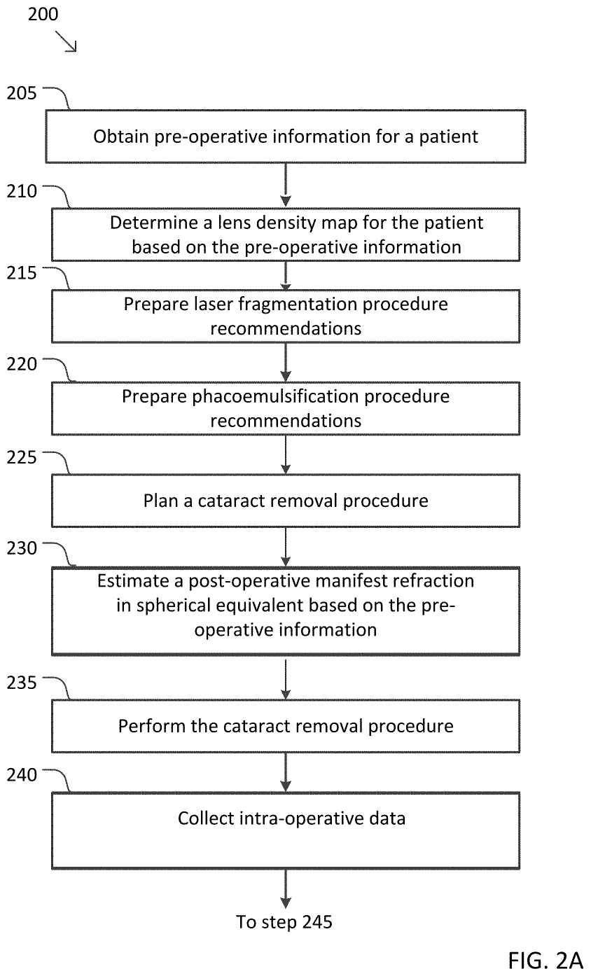

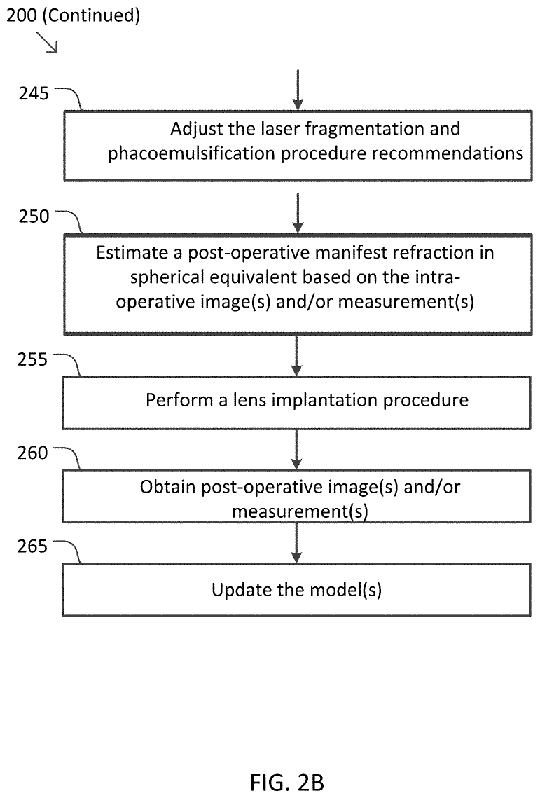

In some examples, the ISP platform 120 provides a custom and optimized surgical plan for a patient to the one or more surgical devices 150. The custom and optimized surgical plan may include recommendations for a laser fragmentation procedure (e.g., further described in relation to process 215 of FIG. 2) as well as recommendations for a phacoemulsification procedure (e.g., further described in relation to process 220 of FIG. 2), among other recommendations. Based on the laser fragmentation and phacoemulsification recommendations, the one or more surgical devices 150 may be configured to (e.g., automatically or in response to surgeon confirmation), provide settings, patterns, targets, and/or feedback (e.g., auditory, optical, and/or haptic feedback) during the surgical procedure.

As an example, these laser fragmentation and phacoemulsification recommendations may include recommended devices settings to be used during each procedure. In certain embodiments, having received the recommended device settings from the ISP platform 150, a surgical device 150 may reconfigure itself after a user, e.g., surgeon, confirms the recommended device settings. In another example, having received the recommended device settings from the ISP platform 150, in certain embodiments, a surgical device 150 may automatically reconfigure itself based on the recommended device settings. Having configured the surgical device 150 with the recommended device settings, the surgical device 150 may be subsequently operated with the recommended device settings by the surgeon to perform a surgery on the corresponding patient.

In addition, in certain embodiments, the surgical device 150 may further provide targets and/or feedback to help the surgeon with following the laser fragmentation and phacoemulsification recommendations (e.g., laser fragmentation patterns, etc.) or help ensure that the surgeon's uses of the surgical device 150 is aligned with the laser fragmentation and phacoemulsification recommendations. For example, the surgical device 150 may use visual indicators on a display of the surgical device 150 (or a connected display, e.g., computing devices 140) to help ensure the surgeon follows the recommended laser fragmentation lines. In another example, feedback may be used to help ensure the surgeon does not apply more laser power than necessary or apply the recommended laser power for longer than necessary.

In some examples, intra-operative data may be collected from surgical devices 150, diagnostic devices 130, etc., and include tracked and/or recorded intra-operative settings, parameters, metrics and/or the like of the one or more surgical devices 150 during the surgical procedure, images and measurements associated with the eye during the procedure, etc. In certain embodiments, the intra-operative data that is collected over the course of the cataract surgery may include or be derived from a surgical video captured during the surgery as well as device log files that capture various sensor I/O parameters from the equipment (e.g., surgical device 150, or any consoles involved) during the surgical procedure. A surgical video can be captured by imaging and camera devices associated with the equipment (e.g., surgical device 150, or any consoles involved) and analyzed using computer vision algorithms and techniques.

The tracked and/or recorded intra-operative settings, patterns, and/or metrics may then be used in multiple ways. For example, the tracked and/or recorded intra-operative settings, patterns, and/or metrics may be used, in real-time, as input into one or more trained models (e.g., fifth one or more models described below in relation to processes 240-245 of FIG. 2) to provide adjusted laser fragmentation and phacoemulsification recommendations. In such an example, by monitoring the real-time conditions relating to the patient's eye during the surgical procedure, intra-operative data can be generated and used to provide dynamic updates to laser fragmentation and phacoemulsification recommendations.

The tracked and/or recorded intra-operative settings, patterns, and/or metrics may be also sent to the ISP platform 105 for use in iteratively training and/or updating the machine learning models (e.g., first and second sets of models described in relation to processes 215 and 220) used by prediction engine 120 so as to incorporate the information from the surgical procedure performed on patient 135 for use in the planning of future surgical procedures. In some cases, the tracked and/or recorded intra-operative settings, patterns, and/or metrics are stored as unstructured or structured data in an ERM database, a cloud-based storage repository, etc.

Example settings, parameters, metrics that may be recorded for each patient (e.g., intraoperatively) include laser fragmentation parameters and metrics (e.g., position and orientation of laser fragmentation lines, distance between laser fragmentation lines (which may be variable), separation distance between laser treatment spots along laser fragmentation lines, use of curved lines (e.g., to trace density contours), use of spiral or other patterns, depth of cuts along each laser fragmentation line, angle of incidence for each fragmentation line (e.g., relative to central axis 480), total time under suction, or other parameters that may be indicative of the features of the laser fragmentation pattern. Example settings, parameters, metrics that may be recorded for each patient may also include laser device settings (e.g., frequency of laser, power level of laser, speed of laser along the laser fragmentation lines, type of laser). Example settings, parameters, metrics that may be recorded for each patient may also include total length of laser cuts (e.g., a total length of the laser fragmentation pattern lines, a total length of time for laser fragmentation, a total laser energy expended, and/or the like).

Example settings, parameters, metrics that may be recorded for each patient (e.g., intraoperatively) may also include phacoemulsification related parameters and metrics (e.g., location of one or more targets within the cataract and/or the lens of the eye where ultrasonic cutting and/or fragmentation energy and/or emulsification are performed, total length of time for phacoemulsification, a total ultrasonic energy, a total volume of applied fluid, total number of laser spots, total ultrasonic energy expended for phacoemulsification, amount of time spent to aspirate the lens, amount of fluid used for aspiration etc.). Example settings, parameters, metrics that may be recorded for each patient may also include phacoemulsification devise settings (e.g., frequency of ultrasonics, power level of ultrasonics, duration of application of ultrasonics, rate and/or volume of fluid to apply, pressure of applied fluid).

The one or more diagnostic devices 130 may further be used to obtain post-operative measurements of patient 135 after the patient undergoes cataract removal and IOL implantation using the selected IOL. The one or more computing devices 140 may then send the post-operative multi-dimensional images and/or measurements of patient 135 and the selected IOL to the ISP platform 105 for use in iteratively training and/or updating the models used by prediction engine 120 so as to incorporate post-operative information associated with patient 135 for use with future patients, as explained in more detail below.

The recommendations provided by a surgical plan may be displayed on one or more computing devices 140 and/or another computing device, display, surgical console, and/or the like. Additionally, the ISP platform 105 and/or the one or more computing devices 140 may identify in the measurements various characteristics of the anatomy of patient 135, as explained below in more detail. Further, the ISP platform 105 and/or the one or more computing devices 140 may create graphical elements that identify, highlight, and/or otherwise depict the patient anatomy, the procedure plan, and/or the measured characteristics for display to the surgeon or other user to further aid in the surgical planning process. The ISP platform 105 and/or the one or more computing devices 140 may supplement the measurements with the graphical elements.

In some embodiments, the ISP platform 105 may further include a surgical planner 160 that creates and provides an optimized surgical plan to ophthalmic practice 125 that uses the recommended patterns and settings for the one or more surgical devices 150 and/or the estimated post-operative MRSEs. In some embodiments, system 100 may further include a stand-alone surgical planner 170 and/or ophthalmic practice 125 may further include a surgical planner module 180 on the one or more computing devices 140 as is described in further detail below.

As discussed above and further emphasized here, FIG. 1 is merely an example which should not unduly limit the scope of the claims. One of ordinary skill in the art would recognize many variations, alternatives, and modifications. According to some embodiments, the ISP platform 105 and/or one or more components thereof, such as databases 155, prediction engine 120, and/or surgical planner 160, may be integrated into the one or more devices of ophthalmic practice 125. In some examples, one or more computing devices 140 may host the ISP platform 105, databases 155, prediction engine 120, and/or surgical planner 160. In some examples, surgical planner 160 may be combined with surgical planner 180.

Note that the collection of the ISP platform 105, at least one of the one or more diagnostic devices 130, at least one of the one or more computing devices 140, at least one of the one or more surgical devices 150 may be referred to as a surgical ophthalmic system that works to implement one or more of the embodiments described herein.

FIGS. 2A-2B show a diagram of a method 200 of removing a cataract according to some embodiments. One or more of the processes 205-265 of method 200 may be implemented, at least in part, in the form of executable code stored on non-transitory, tangible, machine-readable media that when run by one or more processors (e.g., the processors of prediction engine 120, the ISP platform 105, the one or more diagnostic devices 130, the one or more computing devices 140, the one or more surgical devices 150, and/or one or more of the surgical planners 160, 170, and/or 180) may cause the one or more processors to perform one or more of the processes 205-265. According to some embodiments, the process 240 may be performed concurrently with the process 235. According to some embodiments, the process 215 may be performed before the process 210 and/or concurrently with the process 210. Furthermore, the sequence diagram 200 is not required to perform each of or only the shown steps and is not limited to performing the indicated steps in any particular order.

At a process 205, pre-operative information for a patient is obtained. According to some embodiments, the pre-operative information for the patient may include information about the patient, the eye from which a cataract is to be removed, the cataract, and/or the like. For example, in certain embodiments, pre-operative information includes one or more pre-operative images (also referred to as imaging data) and/or one or more pre-operative measurements of an eye. In some examples, the one or more pre-operative images may be extracted from one or more pre-operative images of the eye obtained using a diagnostic device, such as one or more diagnostic devices 130 (e.g., an OCT device, a rotating (e.g., Scheimpflug) camera, an Mill device, a three-dimensional stereoscopic digital microscope (such as NGENUITY® 3D Visualization System (Alcon Inc., Switzerland) and/or the like). In some examples, the one or more pre-operative images may be previously obtained and retrieved from a database (e.g., database 155), storage maintained by ISP platform 105 and/or ophthalmic practice 125, and/or the like.

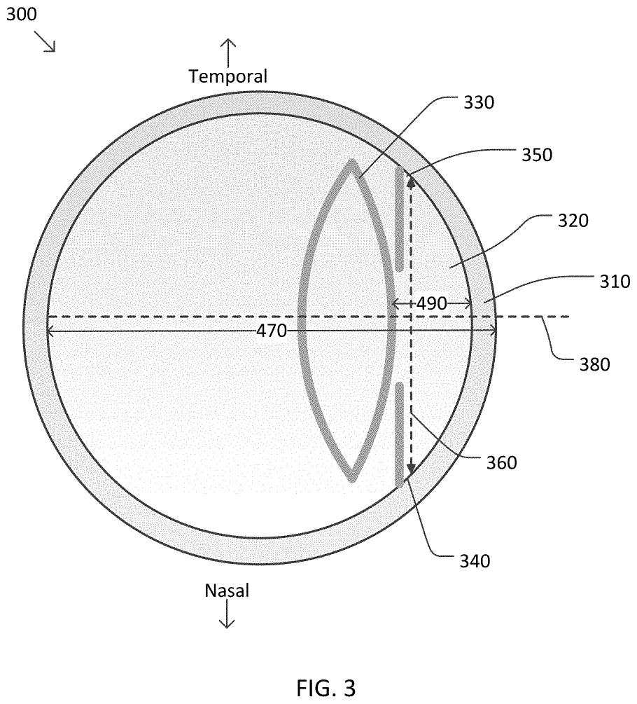

In certain embodiments, one or more pre-operative measurements of the eye may be determined from the one or more pre-operative images. In certain embodiments, one or more of the pre-operative measurements may be determined using one or more measuring devices, such as one or more diagnostic devices 130. The pre-operative measurements of the eye are described herein by reference to FIG. 3, which is a diagram of an eye 300, according to some embodiments. As shown in FIG. 3, eye 300 includes a cornea 310, an anterior chamber 320, and a lens 330.

In some embodiments, one measurement of interest for eye 300 is the white-to-white diameter of cornea 310. In some examples, the white-to-white diameter of cornea 310 may be measured using an optical biometer. In some examples, the white-to-white diameter of cornea 310 may be determined by analyzing the one or more pre-operative images of eye 300. In some examples, the one or more pre-operative images may be analyzed to identify nasal and temporal angles 340 and 350, respectively, of anterior chamber 320. In some examples, nasal and temporal angles 340 and 350 of anterior chamber 320 may be determined from the one or more pre-operative images by (1) identifying structures that are indicative of anterior chamber 320 (e.g., using one or more edge detection and/or region detection algorithms) and (2) noting the acute angles at the edges of anterior chamber 320 located toward the temporal and nasal extents of anterior chamber 320. Once identified, a distance between the nasal and temporal angles 340 and 350 may be measured to determine the white-to-white diameter of cornea 310, which corresponds to a length of line 360 between nasal and temporal angles 340 and 350.

In some embodiments, one measurement of interest for eye 300 is the average keratometry or roundness of the anterior surface of cornea 310. In some examples, the average keratometry of cornea 310 may be measured using the one or more pre-operative images of eye 300, a keratometer, and/or the like. In some examples, the average keratometry of cornea 310 may be based on an average of the steep keratometry and the shallow keratometry measurements of cornea 310. In some examples, the average keratometry of cornea 310 may be expressed as a radius of curvature (rc) of cornea 310, which is 437.5 divided by the average keratometry.

In some embodiments, one measurement of interest from eye 300 is the axial length 370 of eye 300 as measured from the anterior surface of cornea 310 to the retina along central axis 380 of eye 300. In some examples, axial length 370 may be determined using the one or more images of eye 300, biometry of the eye, and/or the like.

In certain embodiments, in addition to the one or more pre-operative images of the patient's eye, the patient history may also be obtained as part of the pre-operative information. In some examples, the patient history may include one or more relevant physiological measurements for the patient that are not directly related to the eye, such as one or more of age, height, weight, body mass index, genetic makeup, race, ethnicity, sex, blood pressure, other demographic and health related information, and/or the like. In some examples, the patient history may further include one or more relevant risk factors including smoking history, diabetes, heart disease, other underlying conditions, prior surgeries, and/or the like and/or a family history for one or more of these risk factors.

At process 210, a lens density map for the eye is determined based on the pre-operative information for the patient (e.g., the one or more pre-operative images). In some examples, the intensity of each pixel and/or voxel from the one or more images may be used to determine the density of a corresponding portion of the lens of the eye captured by the one or more images. Examples of these techniques are described in further detail in commonly owned U.S. Pat. No. 10,314,747 disclosing “Adjusting Laser Energy in Accordance with Optical Density and U.S. Pat. No. 10,433,722 disclosing “Diagnosis System and Diagnosis Method”, which are hereby incorporated by reference in their entireties. In certain embodiments, a type of the cataract (e.g., nucleolus, posterior, anterior cataract) is determined based on the lens density map.

At process 215, one or more recommendations for a laser fragmentation procedure are prepared. In certain embodiments, one or more recommendations for a laser fragmentation procedure are prepared based on the pre-operative information obtained at process 205 and/or the lens density map (including information about the type of cataract) determined at process 210. According to some embodiments, the one or more recommendations may include recommendations for a laser fragmentation pattern to be traced by a laser, such as a femto-second laser, across the cataract and/or the lens of the eye. A laser fragmentation pattern refers to the pattern of laser fragmentation lines to be traced by the laser. In some examples, the recommendations for the laser fragmentation pattern may include one or more of:
Position and orientation of laser fragmentation lines
   E.g., horizontal, vertical, angle
Distance between laser fragmentation lines (which may be variable)
Separation distance between laser treatment spots along laser fragmentation lines
Use of curved lines (e.g., to trace density contours)
Use of spiral or other patterns
Depth of cuts along each fragmentation line
Angle of incidence for each fragmentation line (e.g., relative to central axis 480)
Other parameters associated with one or more features of the laser fragmentation pattern

According to some embodiments, the one or more recommendations may include one or more device settings for the laser device at one or more control points along the laser fragmentation lines. In some examples, the settings may include one or more of:
Frequency of laser
Power level of laser
Speed of laser along the laser fragmentation lines
Type of laser

According to some embodiments, the one or more recommendations may include one or more estimates for the laser fragmentation procedure. In some examples, the one or more estimates may include one or more of a total length of laser cuts (e.g., a total length of the laser fragmentation pattern, a total length of time for laser fragmentation, a total laser energy, and/or the like).

In some examples, a first one or more models, such as one or more of the machine learning models of prediction engine 120, may be used to determine the patterns of the fragmentation lines, the one or more settings, and/or the one or more estimates based on the lens density map and/or combinations of any of the pre-operative information. In some examples, various learning algorithms may be used to train the first one or more models using the training data associated with previous patients, as provided by the diagnostic training data sources 110 and described above. For example, supervised, unsupervised, or other types of machine learning algorithm may be used to train the first one or more models. In some examples, the first one or more models may each include a neural network (e.g., recurrent neural network) trained using the training data.

In certain embodiments, the first one or more models may be trained to determine fragmentation line patterns, settings, and/or estimates that maximize a post-operative survey score indicative of the post-operative surgical outcome. In order to maximize the post-operative survey score the first one or more models may optimize and be trained on features such as time under suction, total laser energy, the number of laser spots, the total length of laser pattern lines, the time required for phacoemulsification, the total ultrasonic energy required for phacoemulsification, time required to aspirate the lens, the amount of fluid required for aspiration, etc.

At a process 220, one or more recommendations for a phacoemulsification procedure are prepared. In certain embodiments, one or more recommendations for a phacoemulsification procedure are prepared based on the lens density map (including information about the type of cataract) and/or combinations of any of the pre-operative information, and/or the recommendations for the laser procedure provided at process 215. According to some embodiments, the one or more recommendations may include one or more recommendations for a location of one or more targets within the cataract and/or the lens of the eye where ultrasonic cutting and/or fragmentation energy and/or emulsification fluid should be applied.

According to some embodiments, the one or more recommendations, may include one or more settings for the phacoemulsification device at each of the targets. In some examples, the one or more settings may include one or more of:
Frequency of ultrasonics
Power level of ultrasonics
Duration of application of ultrasonics
Rate and/or volume of fluid to apply
Pressure of applied fluid

According to some embodiments, the one or more recommendations may include one or more estimates for the phacoemulsification procedure. In some examples, the one or more estimates may include one or more of a total length of time for phacoemulsification, a total ultrasonic energy, a total volume of applied fluid, and/or the like.

In some examples, a second one or more models, such as one or more of the machine learning models of prediction engine 120, may be used to determine the targets for phacoemulsification, the one or more settings, and/or the one or more estimates based on the lens density map and/or combinations of any of the pre-operative information, and/or the recommendations for the laser procedure provided at process 215. In some examples, various learning algorithms may be used to train the second one or more models using the training data associated with previous patients, as provided by the diagnostic training data sources 110 and described above. For example, supervised, unsupervised, or other types of machine learning algorithm may be used to train the second one or more models. In some examples, the second one or more models may each include a neural network (e.g., recurrent neural network) trained using the training data.

In certain embodiments, the second one or more models may be trained to determine phacoemulsification targets, settings, and/or estimates that maximize a post-operative survey score indicative of the post-operative surgical outcome. In order to maximize the post-operative survey score the first one or more models may optimize and be trained on features such as time under suction, total laser energy, the number of laser spots, the total length of laser pattern lines, the time required for phacoemulsification, the total ultrasonic energy required for phacoemulsification, time required to aspirate the lens, the amount of fluid required for aspiration, etc.

At a process 225, a cataract removal procedure is planned. In some examples, the recommendations from processes 215 and/or 220 and/or the pre-operative information obtained during process 205 may be provided to a surgical planner, such as, one or more of surgical planners 160, 170, and/or 180. In some examples, the surgical planner may include a user interface that displays a surgical plan to the surgeon that incudes the recommendations from processes 210 and/or 215 and/or the pre-operative information. For example, the surgical planner may display the laser fragmentation pattern lines and/or the targets determined during processes 210 and 215, respectively, superimposed on one or more images of the eye and/or the cataract (e.g., as obtained during process 205). In some examples, the user interface may further display any of the settings and/or the estimates generated during processes 210 and/or 215. In some examples, the settings may be displayed when the user mouses over and/or clicks on any of the laser fragmentation lines and/or targets. In some examples, the user interface may allow the user to reposition any of the laser fragmentation lines and/or targets and/or change any of the settings.

In some examples, the surgical planner may re-determine any of the recommendations, settings, and estimates based on the changes to the laser-fragmentations lines, targets, and/or settings, such as by repeating portions of processes 215 and/or 220. In certain embodiments, a third one or more models, such as one or more of the machine learning models of prediction engine 120, may be used to re-determine recommendations, settings, and estimates based on changes to the laser-fragmentations lines, targets, and/or settings. In other words, the third one or more models may be trained to take, as input, the changed laser-fragmentations lines, targets, and/or settings, and output laser-fragmentations lines, targets, and/or settings based on the input.

As explained above, a surgeon may have a preferred pattern for phacoemulsification and aspiration of lens material. For example, a surgeon may have been trained to complete phacoemulsification and aspiration of lens material in a certain repeatable pie-slice pattern. The surgeon can be accustomed to emulsifying and removing lens material from a first pie slice and rotating around the lens to subsequent slices to ensure that each area is adequately emulsified and aspirated. Accordingly, one or more of surgical planners 160, 170, and/or 180 can include options for a surgeon or other eye care professional to select a pre-generated or custom made phacoemulsification pattern and a recommended fragmentation pattern that conform with a surgeon's routine, repeatable pattern (e.g., pie-slice pattern).

In addition to a recommended fragmentation pattern, the present technology can include a recommendation for which slices to treat with laser energy, how much laser energy to dedicate to each slice, how much ultrasonic power to deliver to each area of each slice (e.g., based on a predicted ultrasonic power required after the recommended amount of laser energy is delivered to the particular slice), etc. For example, in some cases, a desired phacoemulsification pattern (e.g., pie-slices) can be designated as well as a total laser energy for pre-conditioning the lens. The total laser energy can be selected by the surgeon or other care professional or can be a recommended value based on the historical data processed by the prediction engine 120. For example, the prediction engine 120 can recommend a total laser energy based on a threshold reduction in gas bubble creation and/or a quantified elimination of negative surgical outcome due to gas bubble creation. One or more of surgical planners 160, 170, and/or 180 can use the specified fragmentation pattern, the selected and/or recommended total laser energy, and/or the lens density map to recommend how much laser energy should be applied to the various regions to the cataract to optimize the efficiency of the laser energy in order to pre-condition the most needed areas of the lens for optimal phacoemulsification and aspiration.

In some other cases (sometimes in the absence of a preferred phacoemulsification and aspiration pattern), an optimized surgical plan can recommend a custom fragmentation pattern and device settings based on the lens density map and on various surgical optimization criteria (e.g. reduction of time under suction, reduction of total laser energy, etc.) that are selected by a surgeon and/or recommended by the prediction engine 120, as described above. The one or more surgical planners 160, 170, and/or 180 can recommend using the custom fragmentation pattern and optimization criteria in order to pre-condition the most needed areas of the lens for optimal phacoemulsification and aspiration (even in the absence of preferred phacoemulsification and aspiration pattern).

At a process 230, a post-operative MRSE is estimated for, e.g., each of a given set of IOL powers based on the pre-operative information obtained during process 205. Note that process 250 may be performed before or after process 210. In certain embodiments, a post-operative MRSE may be estimated for each of a plurality of IOL powers that are available on the market based on the patient's pre-operative measurements and/or images (including one or more of the patient's axial length of the eye, corneal curvature, anterior chamber depth, white-to-white diameter of the cornea, lens thickness, an effective lens position (which itself is calculated based on one or more of these pre-operative measurements), etc.). In such embodiments, the surgeon may able to see which one of the IOL powers is estimated to result in a post-operative MRSE that is closest to a desired refractive outcome. In certain other embodiments, a post-operative MRSE may be estimated for a specific IOL power that has been selected by the surgeon. In such embodiments, if the estimated post-operative MRSE is close to a desired refractive outcome, the surgeon may determine that the selected IOL power is likely going to result in a satisfactory refractive outcome for the patient.

Examples of how to use a given IOL power in the estimation of post-operative MRSE are described in further detail in commonly-owned U.S. patent application Ser. No. 16/171,515 filed Oct. 26, 2018 entitled “Systems and Methods for Intraocular Lens Selection,” U.S. Ser. No. 16/746,231, filed Jan. 17, 2020 entitled “Systems and Methods for Intraocular Lens Selection Using Emmetropia Zone Prediction,” and U.S. patent application Ser. No. 16/239,771 filed Jan. 4, 2019 entitled “Systems and Methods for Intraocular Lens Selection,” all of which are hereby incorporated by reference in their entirety. The post-operative MRSE is indicated in diopters (D). In some examples, a fourth one or more models, such as one or more of the models of prediction engine 120, may be used to estimate a post-operative MRSE for, e.g., each of a given set of IOL powers, for a certain patient. In certain embodiments, the fourth one or more models may be trained based on historical patients' pre-operative information (e.g., pre-operative images and/or measurements, patient history, etc.) and post-operative outcomes. For instance, depending on the type of IOL power calculations, example pre-operative measurements used for training the fourth one or more models may include one or more of the patient's axial length of the eye, corneal curvature, anterior chamber depth, white-to-white diameter of the cornea, lens thickness, an effective lens position (which itself is calculated based on one or more of these pre-operative measurements), etc. In some examples, various learning algorithms may be used to train the fourth one or more models using the training data associated with previous patients, as provided by the diagnostic training data sources 110 and described above. For example, supervised, unsupervised, or other types of machine learning algorithm may be used to train the fourth one or more models. In some examples, the fourth one or more models may each include a neural network (e.g., recurrent neural network) trained using the data from the eyes and/or cataract removals of previous patients.

At a process 235, the cataract removal procedure is performed. In some examples, the cataract removal procedure is performed according to the optimized surgical plan provided at process 220. In some examples, a laser may be used to trace the fragmentation pattern using the corresponding one or more settings recommended by process 215. In some examples, when the laser fragmentation is guided by the surgeon, the surgical plan may be used to provide auditory, visual, and/or haptic feedback to help the surgeon guide the laser, as described above. Examples of lasers and laser systems are described in more detail in commonly owned U.S. Pat. No. 9,427,356 disclosing “Photodisruptive Laser Fragmentation of Tissue” and U.S. Pat. No. 9,622,913 disclosing “Imaging-Controlled Laser Surgical System”, both of which are hereby incorporated by reference in their entirety. In some examples, a phacoemulsification device may be used to apply ultrasonic energy to the targets and then use applied fluids to remove the fragmented pieces of the cataract and/or lens. In some examples, when the phacoemulsification is guided by the surgeon, the surgical plan may be used to provide auditory, visual, and/or haptic feedback to help the surgeon guide the phacoemulsification device.

At an optional process 240, intra-operative data is collected. In certain embodiments, intra-operative data refers to settings, parameters, and/or metrics used for the cataract removal procedure. Process 240 may be performed concurrently with process 235 such that as the cataract removal procedure is being performed during process 235, one or more settings, parameters, and metrics are tracked and recorded.

In certain embodiments, intra-operative data that is collected over the course of the cataract surgery may include or be derived from a surgical video captured during the surgery as well as device log files that capture various sensor input/output parameters from the equipment (e.g., surgical device 150, or any consoles involved) during the surgical procedure. A surgical video can be captured by imaging and camera devices associated with the equipment (e.g., surgical device 150, or any consoles involved) and analyzed using computer vision algorithms and techniques. The intra-operative data collected may include any data point or metric relating to inputs and outputs of the models described herein. For example, intra-operative data may include time-stamped eye-related information, such as changes to any aspect (e.g., tissues, lens, other components, etc.) of the eye as the procedure is being performed, time-stamped settings, parameters, metrics collected during the procedure (e.g., laser fragmentation and/or phacoemulsification).

Example settings, parameters, metrics that may be recorded for each patient include laser fragmentation parameters and metrics (e.g., position and orientation of laser fragmentation lines, distance between laser fragmentation lines (which may be variable), separation distance between laser treatment spots along laser fragmentation lines, use of curved lines (e.g., to trace density contours), use of spiral or other patterns, depth of cuts along each laser fragmentation line, angle of incidence for each fragmentation line (e.g., relative to central axis 480), total time under suction, or other parameters that may be indicative of the features of the laser fragmentation pattern. Example settings, parameters, metrics that may be recorded for each patient may also include laser device settings (e.g., frequency of laser, power level of laser, speed of laser along the laser fragmentation lines, type of laser). Example settings, parameters, metrics that may be recorded for each patient may also include total length of laser cuts (e.g., a total length of the laser fragmentation pattern lines, a total length of time for laser fragmentation, a total laser energy expended, and/or the like).

Example settings, parameters, metrics that may be recorded for each patient may also include phacoemulsification related parameters and metrics (e.g., location of one or more targets within the cataract and/or the lens of the eye where ultrasonic cutting and/or fragmentation energy and/or emulsification are performed, total length of time for phacoemulsification, a total ultrasonic energy, a total volume of applied fluid, total number of laser spots, total ultrasonic energy expended for phacoemulsification, amount of time spent to aspirate the lens, amount of fluid used for aspiration etc.). Example settings, parameters, metrics that may be recorded for each patient may also include phacoemulsification devise settings (e.g., frequency of ultrasonics, power level of ultrasonics, duration of application of ultrasonics, rate and/or volume of fluid to apply, pressure of applied fluid).

In certain embodiments, the intra-operative data may include one or more intra-operative images and/or measurements. The one or more intra-operative images and/or measurements may include images and/or measurements of the eye as the procedure is being performed, prior to the lens being completely removed. The one or more intra-operative images and/or measurements may also include intra-operative images and/or measurements of an aphakic eye. For example, an intra-operative optical measurement device 130 (e.g., the Ora™ with Verifeye™ (Alcon Inc., Switzerland) is used to provide intra-operative measurements of the eye, including one or more of the curvature of the cornea, axial length of the eye, white-to-white diameter of the cornea, etc.

At an optional process 245, the laser fragmentation procedure recommendations and/or the phacoemulsification procedure recommendations are adjusted based on the intra-operative data collected at optional process 240. Process 245 may be performed concurrently with process 235 and 240. For example, the intra-operative data may be provided as input into a fifth one or more models to provide adjusted recommendations. Adjusting the recommendations provided by processes 215 and 220 may be advantageous because the collected intra-operative data may make such recommendations sub-optimal. For example, in certain cases, intra-operative images associated with the eye may provide data points that were unknown pre-operatively and or not entirely accurate. In addition, the recommendations provided by processes 215 and 220 may impact the patient's eye in ways that were not anticipated. Also, a surgeon may not fully follow some of the recommendations provided by processes 215 and 220, causing the rest of the recommendations provided by processes 215 and 220 sub-optimal or useless. Therefore, the fifth one or more models may continuously and periodically take the time-stamped intra-operative data as input during the procedure and provide adjusted or updated laser fragmentation procedure recommendations and/or the phacoemulsification procedure recommendations.

The fifth one or more models may include one or more reinforcement leaning models. Reinforcement learning (RL) is an area of machine learning concerned with designing intelligent agents that are responsive to changes in a real-world situation and can take actions in order to maximize the notion of a cumulative reward. An intelligent agent includes (A) a policy and (B) an algorithm (e.g., reinforcement learning algorithm) for updating the policy. The policy is a model (e.g., sometimes a deep neural network or a simpler supervised learning model) that decides what action to take (i.e., what recommendations to provide in terms of parameters, settings, and metrics used during the procedure) given a set of state observations (i.e., the state of the environment as it pertains to the eye and the surgical devices being used). In other words, the policy is the brain of the agent that takes in state observations and maps them to actions. The RL algorithm updates the policy, as the policy may not be mapped correctly to take the best action or the environment (e.g., defined by all the data points derived from the intra-operative data discussed above) may change, making the mapping not optimal. The RL algorithm changes the policy based on the actions that were taken, the observations from the environment, and the amount of reward collected, as determined by a reward function, described below. Using the RL algorithm, the agent, therefore, modifies its policy as it interacts with the environment so that, eventually, given any state, it will always take the most advantageous action that corresponds to the most reward in the long run.

At a process 250, a post-operative MRSE is estimated for, e.g., each of a given set of IOL powers based on the intra-operative information obtained during process 245. In certain embodiments, a post-operative MRSE may be estimated for each of a plurality of IOL powers that are, for example, available on the market. In such embodiments, the surgeon may able to see which one of the IOL powers is estimated to result in a post-operative MRSE that is closest to a desired refractive outcome. In certain other embodiments, a post-operative MRSE may be estimated for a specific IOL power that has been selected by the surgeon. In such embodiments, if the estimated post-operative MRSE is close to a desired refractive outcome, the surgeon may determine that the selected IOL power is likely going to result in a satisfactory refractive outcome for the patient.

In certain embodiments, one or more post-operative MRSEs are intra-operatively estimated for the patient based the patient's aphakic measurements including on one or more of the axial length, corneal curvature, anterior chamber depth, white-to-white diameter of the cornea, lens thickness, an effective lens position. In some examples, the one or more post-operative MRSEs calculated based on the patient's intra-operative measurements at process 250 may be different than the one or more post-operative MRSEs calculated based on the patient's pre-operative measurements at process 230. In such examples, the surgeon may select an IOL power based on the one or more post-operative MRSEs calculated using the patient's intra-operative measurements and ignore a previously selected IOL power. Performing intra-operative measurements using a device, such as the Ora™ with Verifeye™ (Alcon Inc., Switzerland), is therefore advantageous for ensuring that an optimal IOL power is used, resulting in a satisfactory refractive outcome.

In certain embodiments, the sixth one or more models may be trained based on historical patients' intra-operative information and post-operative outcomes. In some examples, various learning algorithms may be used to train the sixth one or more models using the training data associated with previous patients, as provided by the diagnostic training data sources 110 and described above. For example, supervised, unsupervised, or other types of machine learning algorithm may be used to train the sixth one or more models. In some examples, the sixth one or more models may each include a neural network trained (e.g., recurrent neural network) using the data from the eyes and/or cataract removals of previous patients.

At process 255, a lens implantation procedure is performed for implanting an IOL with the selected IOL power to replace the fragmented and removed lens.

At an optional process 260, one or more post-operative measurements of the eye are obtained and/or a post-operative satisfaction score is recorded. In some examples, the one or more post-operative measurements may include an actual post-operative MRSE after implantation of the IOL during process 255 and/or the like. In some examples, the actual post-operative MRSE may be determined based on one or more images of the post-operative eye, one or more physiological and/or optical measurements of the post-operative eye, and/or the like.

At a process 265, the first, second, third, fourth, fifth and/or sixth sets of models used by method 200 are updated. In some examples, the pre-operative information determined during process 205, the lens density map determined at process 210, the settings, parameters, and metrics recorded during process 240, the one or more intra-operative measurements obtained during process 245, the one or more post-operative measurements obtained during process 260, and/or the like may be used as additional training data for any of the first, second third, fourth, fifth, and/or sixth sets of models. In some examples, the additional training data may be added to a data source, such as data source 110. In some examples, the updating may include one or more of updating least-squares fits, feedback to neural networks (e.g., using back propagation), and/or the like.

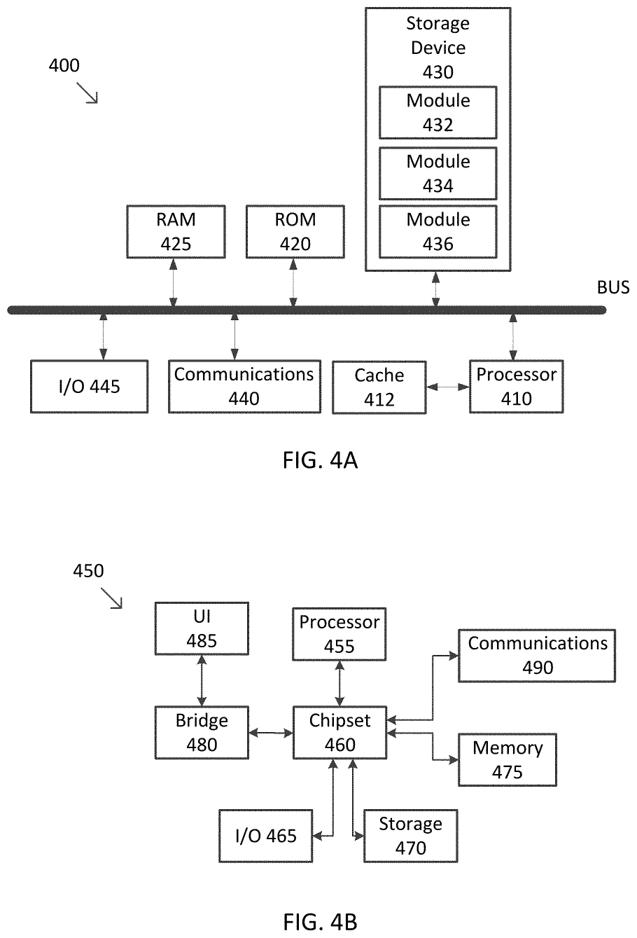

FIGS. 4A and 4B are diagrams of processing systems according to some embodiments. Although two embodiments are shown in FIGS. 4A and 4B, persons of ordinary skill in the art will also readily appreciate that other system embodiments are possible. According to some embodiments, the processing systems of FIGS. 4A and/or 4B are representative of computing systems that may be included in one or more of IOL selection and procedure planning platform 105, ophthalmic practice 125, prediction engine 120, one or more diagnostic devices 130, the one or more computing devices 140, any of surgical planner 160, 170, and/or 180, and/or the like.

FIG. 4A illustrates a computing system 400 where the components of system 400 are in electrical communication with each other using a bus 405. System 400 includes a processor 410 and a system bus 405 that couples various system components including memory in the form of a read only memory (ROM) 420, a random access memory (RAM) 425, and/or the like (e.g., PROM, EPROM, FLASH-EPROM, and/or any other memory chip or cartridge) to processor 410. System 400 may further include a cache 412 of high-speed memory connected directly with, in close proximity to, or integrated as part of processor 410. System 400 may access data stored in ROM 420, RAM 425, and/or one or more storage devices 430 through cache 412 for high-speed access by processor 410. In some examples, cache 412 may provide a performance boost that avoids delays by processor 410 in accessing data from memory 415, ROM 420, RAM 425, and/or the one or more storage devices 430 previously stored in cache 412. In some examples, the one or more storage devices 430 store one or more software modules (e.g., software modules 432, 434, 436, and/or the like). Software modules 432, 434, and/or 436 may control and/or be configured to control processor 410 to perform various actions, such as the processes of methods 200 and/or 300. And although system 400 is shown with only one processor 410, it is understood that processor 410 may be representative of one or more central processing units (CPUs), multi-core processors, microprocessors, microcontrollers, digital signal processors (DSPs), field programmable gate arrays (FPGAs), application specific integrated circuits (ASICs), graphics processing units (GPUs), tensor processing units (TPUs), and/or the like. In some examples, system 400 may be implemented as a stand-alone subsystem and/or as a board added to a computing device or as a virtual machine.

To enable user interaction with system 400, system 400 includes one or more communication interfaces 440 and/or one or more input/output (I/O) devices 445. In some examples, the one or more communication interfaces 440 may include one or more network interfaces, network interface cards, and/or the like to provide communication according to one or more network and/or communication bus standards. In some examples, the one or more communication interfaces 440 may include interfaces for communicating with system 400 via a network, such as network 115. In some examples, the one or more I/O devices 445 may include on or more user interface devices (e.g., keyboards, pointing/selection devices (e.g., mice, touch pads, scroll wheels, track balls, touch screens, and/or the like), audio devices (e.g., microphones and/or speakers), sensors, actuators, display devices, and/or the like).

Each of the one or more storage devices 430 may include non-transitory and non-volatile storage such as that provided by a hard disk, an optical medium, a solid-state drive, and/or the like. In some examples, each of the one or more storage devices 430 may be co-located with system 400 (e.g., a local storage device) and/or remote from system 400 (e.g., a cloud storage device).

FIG. 4B illustrates a computing system 450 based on a chipset architecture that may be used in performing any of the methods (e.g., methods 200 and/or 300) described herein. System 450 may include a processor 455, representative of any number of physically and/or logically distinct resources capable of executing software, firmware, and/or other computations, such as one or more CPUs, multi-core processors, microprocessors, microcontrollers, DSPs, FPGAs, ASICs, GPUs, TPUs, and/or the like. As shown, processor 455 is aided by one or more chipsets 460, which may also include one or more CPUs, multi-core processors, microprocessors, microcontrollers, DSPs, FPGAs, ASICs, GPUs, TPUs, co-processors, coder-decoders (CODECs), and/or the like. As shown, the one or more chipsets 460 interface processor 455 with one or more of one or more I/O devices 465, one or more storage devices 470, memory 475, a bridge 480, and/or one or more communication interfaces 490. In some examples, the one or more I/O devices 465, one or more storage devices 470, memory, and/or one or more communication interfaces 490 may correspond to the similarly named counterparts in FIG. 4A and system 400.

In some examples, bridge 480 may provide an additional interface for providing system 450 with access to one or more user interface (UI) components, such as one or more keyboards, pointing/selection devices (e.g., mice, touch pads, scroll wheels, track balls, touch screens, and/or the like), audio devices (e.g., microphones and/or speakers), display devices, and/or the like.

According to some embodiments, systems 400 and/or 460 may provide a graphical user interface (GUI) suitable for aiding a user (e.g., a surgeon and/or other medical personnel) in the performance of the processes of methods 200 and/or 300. The GUI may include depictions of editable surgical plans, instructions regarding the next actions to be performed, diagrams of annotated and/or un-annotated anatomy, such as pre-operative and/or post-operative images of an eye (e.g., such as depicted in FIG. 4), requests for input, and/or the like. In some examples, the GUI may display true-color and/or false-color images of the anatomy, and/or the like.

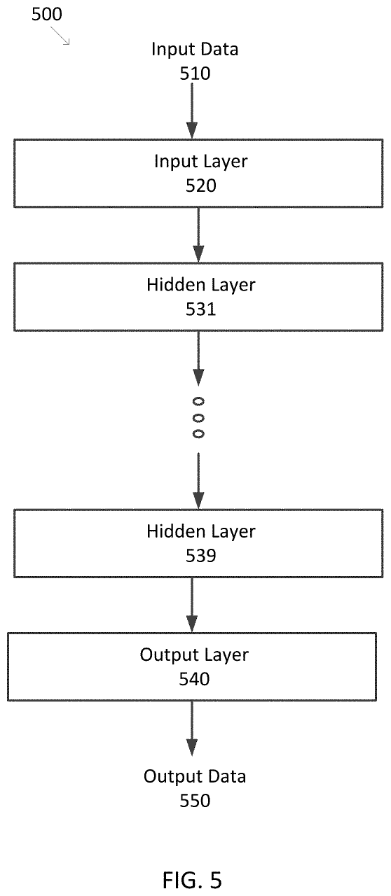

FIG. 5 is a diagram of a multi-layer neural network 500 according to some embodiments. In some embodiments, neural network 500 may be representative of a neural network used to implement each of the first, second, third, fourth, fifth and sixth sets of models as well as any other models described herein (e.g., with respect to method 200 and used by prediction engine 120). Neural network 500 processes input data 510 using an input layer 520. In some examples, input data 510 may correspond to the input data (e.g., data provided by the training data source(s) 110) provided to the one or more models and/or the training data provided to the one or more models, e.g., during the updating during process 265 used to train the one or more models. Input layer 520 includes a plurality of neurons that are used to condition input data 510 by scaling, range limiting, and/or the like. Each of the neurons in input layer 520 generates an output that is fed to the inputs of a hidden layer 531. Hidden layer 531 includes a plurality of neurons that process the outputs from input layer 520. In some examples, each of the neurons in hidden layer 531 generates an output that are then propagated through one or more additional hidden layers that end with hidden layer 539. Hidden layer 539 includes a plurality of neurons that process the outputs from the previous hidden layer. The outputs of hidden layer 539 are fed to an output layer 540. Output layer 540 includes one or more neurons that are used to condition the output from hidden layer 539 by scaling, range limiting, and/or the like. It should be understood that the architecture of neural network 500 is representative only and that other architectures are possible, including a neural network with only one hidden layer, a neural network without an input layer and/or output layer, a neural network with recurrent layers, and/or the like.

In some examples, each of input layer 520, hidden layers 531-539, and/or output layer 540 includes one or more neurons. In some examples, each of input layer 520, hidden layers 531-539, and/or output layer 540 may include a same number or a different number of neurons. In some examples, each of the neurons takes a combination (e.g., a weighted sum using a trainable weighting matrix W) of its inputs x, adds an optional trainable bias b, and applies an activation function f to generate an output a as shown in Equation 1. In some examples, the activation function f may be a linear activation function, an activation function with upper and/or lower limits, a log-sigmoid function, a hyperbolic tangent function, a rectified linear unit function, and/or the like. In some examples, each of the neurons may have a same or a different activation function.

a=f(Wx+b) Equation 1

In some examples, neural network 500 may be trained using supervised learning (e.g., during process 265) where combinations of training data that include a combination of input data and a ground truth (e.g., expected) output data. Differences between the output of neural network 500 as generated using the input data for input data 510 and comparing output data 550 as generated by neural network 500 to the ground truth output data. Differences between the generated output data 550 and the ground truth output data may then be fed back into neural network 500 to make corrections to the various trainable weights and biases. In some examples, the differences may be fed back using a back propagation technique using a stochastic gradient descent algorithm, and/or the like. In some examples, a large set of training data combinations may be presented to neural network 500 multiple times until an overall loss function (e.g., a mean-squared error based on the differences of each training combination) converges to an acceptable level.

As described above, one example of a neural network that may be used as part of the first, second, third, fourth, fifth, and sixth set of models may be a recurrent neural network (RNN). An RNNs is a type of neural network model that can learn from temporal data. RNNs have multiple connected neural networks that are connected through an internal state that can preserve temporal information. Process of training such an RNN model may include, but not limited to, setting up a training dataset from prior patients to train the model (training dataset may include a combination of patient pre-op, intra-op and post-op info), setting up a success criteria by formulating the “loss function which optimized all relevant surgical parameters of interest” so as to provide a good post-op outcome (e.g., maximize the post-op survey score), customizing and using a Long Short Term Memory (LSTM) model, a type of RNN, that may apply backpropagation and other optimization techniques to “objectify surgical tasks” and learn “optimal parameter settings” from the intra-op data.

Methods according to the above-described embodiments may be implemented as executable instructions that are stored on non-transitory, tangible, machine-readable media. The executable instructions, when run by one or more processors (e.g., processor 510 and/or process 555) may cause the one or more processors to perform one or more of the processes of methods 200 and/or 300. Some common forms of machine-readable media that may include the processes of methods 200 and/or 300 are, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, and/or any other medium from which a processor or computer is adapted to read.

Devices implementing methods according to these disclosures may comprise hardware, firmware, and/or software, and may take any of a variety of form factors. Typical examples of such form factors include laptops, smart phones, small form factor personal computers, personal digital assistants, and/or the like. Portions of the functionality described herein also may be embodied in peripherals and/or add-in cards. Such functionality may also be implemented on a circuit board among different chips or different processes executing in a single device, by way of further example.

Although illustrative embodiments have been shown and described, a wide range of modification, change and substitution is contemplated in the foregoing disclosure and in some instances, some features of the embodiments may be employed without a corresponding use of other features. One of ordinary skill in the art would recognize many variations, alternatives, and modifications. Thus, the scope of the invention should be limited only by the following claims, and it is appropriate that the claims be construed broadly and in a manner consistent with the scope of the embodiments disclosed herein.

What is claimed is:

1. A method for use in relation to removing a lens of an eye, comprising:
obtaining pre-operative data for the eye, the pre-operative data including imaging data associated with the lens of the eye;
determining a lens density map based on the imaging data associated with the lens; and
generating laser fragmentation patterns for a laser fragmentation procedure based on the lens density map.

2. The method of claim 1, determining the lens density map comprises analyzing an intensity of each of a plurality of pixels or each of a plurality of voxels from the imaging data.

3. The method of claim 1, further comprising determining a type of cataract based on the lens density map, wherein generating the laser fragmentation patterns is further based on the type of cataract.

4. The method of claim 1, further comprising generating one or more device settings for a laser device used for performing the laser fragmentation procedure.

5. The method of claim 4, wherein the one or more device settings comprise a frequency of laser, a power of laser, a speed of laser, or a type of laser.

6. The method of claim 1, wherein the generated laser fragmentation patters indicate at least one of a position and orientation of fragmentation lines, a distance between the fragmentation lines, a separation distance between laser treatment spots along the fragmentation lines, a use of curved lines, a use of spiral or irregular patterns, a depth of cuts along each of the fragmentation lines, or an angle of incidence for each pattern line relative to central axis.

7. The method of claim 1, wherein the generating further comprises generating at least one of a total length of fragmentation lines associated with the laser fragmentation pattern, a total length of time for the laser fragmentation procedure, or a total laser energy used for the laser fragmentation procedure.

8. The method of claim 1, wherein the generating is based on at least one of optimizing a time under suction associated with the laser fragmentation procedure, optimizing a total laser energy expended for the laser fragmentation procedure, optimizing a number of laser spots, optimizing of the total length of laser fragmentation lines, optimizing a time required for phacoemulsification, optimizing a total ultrasonic energy required for phacoemulsification, optimizing a time required to aspirate the lens, optimizing an amount of fluid required for aspiration.

9. The method of claim 1, further comprising:
obtaining intra-operative data collected while the lens is fragmented; and
adjusting the laser fragmentation patterns based on the intra-operative data.

10. The method of claim 1, wherein the generating is based on maximizing a predicted post-operative survey score based on historical post-operative survey scores.

11. The method of claim 1, wherein the generating further comprises identifying one or more locations of one or more corresponding targets associated with the lens for applying ultrasonic energy to.

12. The method of claim 11, wherein the generating further comprises generating one or more phacoemulsification device settings for each of the one or more corresponding targets.

13. The method of claim 11, wherein the one or more phacoemulsification device settings include at least one of a frequency of an ultrasonic device, or a power level of the ultrasonic device, duration of application of ultrasonics, rate and/or volume of fluid to apply, or pressure of applied fluid.

14. An ophthalmic system used in relation to removing a lens of an eye, comprising:
at least one memory comprising executable instructions;
at least one processor in data communication with the at least one memory and configured to execute the instructions to cause the ophthalmic system to:
obtain pre-operative data for the eye, the pre-operative data including imaging data associated with the lens of the eye;
determine a lens density map based on the imaging data associated with the lens; and
generate laser fragmentation patterns for a laser fragmentation procedure based on the lens density map.

15. The ophthalmic system of claim 14, wherein the processor being configured to cause the ophthalmic system to determine the lens density map comprises the processor being configured to cause the ophthalmic system to analyze an intensity of each of a plurality of pixels or each of a plurality of voxels from the imaging data.

16. The ophthalmic system of claim 14, wherein:
the processor is further configured to cause the ophthalmic system to determine a type of cataract based on the lens density map, and
processor being configured to generate the laser fragmentation patterns is further based on the type of cataract.

17. A non-transitory computer readable medium having instructions stored thereon that, when executed by an ophthalmic system, cause the ophthalmic system to perform a method comprising:
obtaining pre-operative data for the eye, the pre-operative data including imaging data associated with the lens of the eye;
determining a lens density map based on the imaging data associated with the lens; and
generating laser fragmentation patterns for a laser fragmentation procedure based on the lens density map.

18. The non-transitory computer readable medium of claim 17, wherein determining the lens density map comprises analyzing an intensity of each of a plurality of pixels or each of a plurality of voxels from the imaging data.

19. The non-transitory computer readable medium of claim 17, wherein the method further comprises determining a type of cataract based on the lens density map, and wherein generating the laser fragmentation patterns is further based on the type of cataract.
